(19) United States
(12) Patent Application Publication
(10) Pub. No.: US 2007/0038096 A1
(43) Pub. Date: Feb. 15, 2007

(54) Method of optimizing an ultrasound transducer

(76) Inventors: Ralf Seip, Indianapolis, IN (US); Narendra T. Sanghvi, Indianapolis, IN (US); Artur P. Katny, Ingalls, IN (US); Dennis J. Shaver, New Ross, IN (US)

Correspondence Address: BAKER & DANIELS LLP, 300 NORTH MERIDIAN STREET, SUITE 2700, INDIANAPOLIS, IN, 46204, US

(21) Appl. No.: 11/175,947

(22) Filed: Jul. 6, 2005

(51) Int. Cl. A61B 8/00

(52) U.S. Cl. 600/439

(57) ABSTRACT

Methods and apparatuses are disclosed to optimize an acoustic transducer, such as an ultrasound transducer, for use in a therapy application, such as high intensity focused ultrasound. In one example, the thickness of a matching layer applied to a face of a transducer member is altered to optimize the transducer for high intensity focused ultrasound operation at a desired frequency based on total power measurements as a function of acoustic frequency and lapping iterations.

### Background of the Invention

The present invention relates methods and apparatuses for optimizing an acoustic transducer, and in particular to optimize an ultrasound transducer used to provide high intensity focused ultrasound (“HIFU”) therapy to tissue and/or to image tissue.

The treatment of tissue with HIFU energy is known in the art. For instance, HIFU may be used in the treatment of benign prostatic hyperplasia (BPH) and prostate cancer (PC). Further, it is know to image tissue with an ultrasound transducer. In addition, it is known to use a two-element transducer to both image the tissue to be treated with HIFU or that has been treated with HIFU and to provide the actual treatment with HIFU.

An exemplary system for treating the prostate with HIFU is the Sonablate®-500 system available from Focus Surgery located at 3940 Pendleton Way, Indianapolis, Ind. 46226. The Sonablate 500 system uses a dual-element, confocal ultrasound transducer which is moved by mechanical methods, such as motors, under the control of a controller. Typically one element of the transducer, the central element, is used for imaging and both elements (the central and outer elements) of the transducer are used for providing HIFU therapy to the tissue to be treated.

Transducers, such as ultrasound transducers, include a transducer member, such as a piezo-electric crystal, which generates and/or detects acoustic energy. Both the transducer member and the surrounding environment have an associated acoustic impedance. Assuming that the acoustic impedance of transducer member is generally the same as the acoustic impedance of the surrounding environment, acoustic energy would flow from the transducer member to the surrounding environment generally without loss of acoustic energy. However, there is often a difference between the acoustic impedance of the transducer member and the acoustic impedance of the surrounding environment. This mismatch results in less acoustic energy being transferred from the transducer member to the surrounding environment. The reduction in transfer of acoustic energy results in the generation of heat associated with the transducer member which may lead to damage to transducer member or to the surrounding environment. Further, the reduction in transfer of acoustic energy results in a higher level of acoustic energy required to provide sufficient acoustic energy at a treatment site in the surrounding environment.

It is known to have an acoustical matching layer applied to the front surface of the transducer member to reduce the acoustic impedance mismatch between transducer member and the surrounding environment. By reducing the acoustic impedance mismatch, less energy is required to provide therapy and less heat is generated at the transducer. Generally the acoustical matching layer has an acoustic impedance between the acoustic impedance of transducer member and the acoustic impedance of the surrounding environment.

The thickness of the matching layer is one factor in the performance of the transducer. Two known traditional methods used in the manufacture of transducers to make sure an appropriate thickness matching layer is present include the use of thickness gauges to measure the thickness of the matching layer at various positions of the transducer surface and the monitoring of the shape of an echo pulse received based on acoustic pulse emitted by the transducer is monitored.

Transducers used in imaging applications typically operate at acoustic power levels of a few milliwatts. In contrast, transducers used for therapy applications are required to emit higher amounts of acoustic energy than for traditional imaging applications, such as in the range of about 5 to more than 100 Watts. As such, an inefficient coupling between the transducer member for a therapy application and the surrounding environment results in a greater generation of heat and potential damage to the transducer and/or the surrounding environment. A need exists for a method and apparatus to optimize an acoustic transducer for therapy applications. A further need exists for a method and apparatus to optimize an acoustic transducer for therapy applications and to improve the imaging capabilities of the acoustic transducer at the same time.

### Summary of the Invention

In an exemplary embodiment of the present invention a method for optimizing an ultrasound transducer for therapy applications is provided. In one example, the ultrasound transducer is optimized for providing high intensity focused ultrasound during a therapy application.

In another exemplary embodiment of the present invention, a method of optimizing an ultrasound transducer to provide therapy with HIFU at a desired frequency is provided. The method comprising the steps of: driving an ultrasound transducer at a plurality of acoustic frequencies; detecting an acoustic power of the ultrasound transducer for each of the plurality of acoustic frequencies; and altering a thickness of a matching layer of the ultrasound transducer based on the detected acoustic power of the ultrasound transducer. In an example, the step of altering the thickness of the matching layer is repeated until a maximum detected acoustic power is at the desired frequency. In another example, the step of altering the thickness of the matching layer is repeated until a stored threshold power is exceeded by the detected acoustic power at the desired frequency. In a further example, the step of detecting the acoustic power of the ultrasound transducer includes the steps of: placing the ultrasound transducer in a test tank filled with a test substance; placing an acoustic absorber in a position to receive the acoustic energy of the ultrasound transducer; and sensing the acoustic power of the ultrasound transducer for each acoustic frequency with the acoustic absorber to determine a total acoustic power output of the ultrasound transducer for each acoustic frequency. In yet a further example, the ultrasound transducer includes a central transducer element and an outer transducer element and wherein the method further comprises the step of applying a backing to at least the central transducer element. In one variation, the method further comprises the step of adjusting one of a thickness of the backing and a density of the backing to optimize the central element for an imaging mode of operation.

In a further exemplary embodiment of the present invention, a method of optimizing an ultrasound transducer to provide therapy with HIFU at a desired frequency is provided. The method comprising the steps of: receiving an indication of an acoustic power of the ultrasound transducer across a range of acoustic frequencies, including the desired frequency; and altering a thickness of a matching layer of the ultrasound transducer until a maximum of the acoustic power of the ultrasound transducer across the range of acoustic frequencies corresponds to the desired frequency. In an example, the method further comprises the step of applying the matching layer to a face of a transducer member of the ultrasound transducer, the matching layer having an initial thickness greater than a final optimized thickness. In one variation, the step of altering the thickness of the matching layer comprises the steps of: (a) lapping a face of the matching layer to reduce the thickness of the matching layer; (b) receiving an updated indication of the acoustic power of the ultrasound transducer across the range of acoustic frequencies; and (c) repeating steps (a) and (b) until the maximum of the acoustic power of the ultrasound transducer corresponds to the desired frequency. In another example, the indication of the acoustic power of the ultrasound transducer is received by the steps of: placing the ultrasound transducer in a test tank filled with a test substance; placing an acoustic absorber in a position to receive the acoustic energy of the ultrasound transducer; exciting the ultrasound transducer to generate a plurality of acoustic frequencies, each at a respective period of time; and sensing the acoustic power of the ultrasound transducer for each acoustic frequency with the acoustic absorber to determine a total acoustic power output of the ultrasound transducer for each acoustic frequency. In a further example, the indication of the acoustic power of the ultrasound transducer is determined by the step of monitoring an impedance associated with the ultrasound transducer. In still a further example, the ultrasound transducer includes a central transducer element and an outer transducer element and wherein the method further comprises the step of applying a backing to at least the central transducer element. In one variation, method further comprises the step of adjusting one of a thickness of the backing and a density of the backing to optimize the central element for an imaging mode of operation.

In still a further exemplary embodiment of the present invention, a method of optimizing an ultrasound transducer to provide therapy with HIFU at a desired frequency is provided. The method comprising the steps of: (a) detecting a plurality of acoustic powers of the ultrasound transducer, each acoustic power corresponding to a given frequency in a range of acoustic frequencies, including the desired frequency; (b) altering a thickness of a matching layer of the ultrasound transducer; and (c) repeating steps (a) and (b) until a detected power corresponding to the desired frequency for a current iteration is less than a detected power corresponding to the desired frequency for a prior iteration. In an example, steps (a) and (b) are repeated until the detected power corresponding to the desired frequency for the current iteration is less than the detected power corresponding to the desired frequency for the prior iteration and the detected power corresponding to the desired frequency for the current iteration exceeds a threshold power level. In another example, steps (a) and (b) are repeated until the detected power corresponding to the desired frequency for the current iteration is less than the detected power corresponding to the desired frequency for the prior iteration, the detected power corresponding to the desired frequency for the current iteration exceeds the threshold power level, and the detected power corresponding to the desired frequency for the current iteration is equal to a maximum detected power for the current iteration. In a further example, the method further comprises the step of applying the matching layer to a face of a transducer member of the ultrasound transducer. The matching layer having an initial thickness greater than a final thickness. In still a further example, the step of detecting a plurality of acoustic powers of the ultrasound transducer includes the steps of: placing the ultrasound transducer in a test tank filled with a test substance; placing an acoustic absorber in a position to receive the acoustic energy the ultrasound transducer; exciting the ultrasound transducer to generate a plurality of acoustic frequencies, each at a respective period of time; and sensing the acoustic power of the ultrasound transducer for each given acoustic frequency with the acoustic absorber to determine a total acoustic power output of the ultrasound transducer for each given acoustic frequency. In yet another example, the transducer includes a central transducer element and an outer transducer element and wherein the method further comprises the step of applying a backing to at least the central transducer element. In one variation, the method further comprises the step of adjusting one of a thickness of the backing and a density of the backing to optimize the central element for an imaging mode of operation.

In yet another exemplary embodiment of the present invention, a computer readable medium providing instructions for directing a controller is provided, The computer readable medium providing instructions directing the controller to drive an ultrasound transducer at a plurality of acoustic frequencies; detect an acoustic power of the ultrasound transducer for each of the plurality of acoustic frequencies; and provide an indication that a thickness of a matching layer of the ultrasound transducer be altered based on the detected acoustic power of the ultrasound transducer. In an example, the indication is provided until a maximum detected acoustic power is at a frequency which is the desired frequency. In another example, the indication is provided until a stored threshold power is exceeded by the detected acoustic power at the desired frequency. In a further example, the computer readable medium further provides instructions to provide a second indication that transducer is optimized for a therapy application.

Additional features of the present invention will become apparent to those skilled in the art upon consideration of the following detailed description of the illustrative embodiment exemplifying the best mode of carrying out the invention as presently perceived.

### Brief Description of the Drawings

The detailed description of the drawings particularly refers to the accompanying figures in which:

FIG. 1 is a representative view of an exemplary multiple element transducer with an exemplary matching layer exploded therefrom and an exemplary control system;

FIG. 2 is a representative sectional view of the multiple element transducer of FIG. 1 along lines 2-2 in FIG. 1, illustrating a final thickness of the matching layer, an initial thickness of the matching layer, and an intermediate thickness of the matching layer;

FIG. 2A is a representative sectional view of an exemplary transducer with an exemplary matching layer, the transducer having multiple elements defined by the placement of electrodes on a convex side of the transducer;

FIG. 3 is a flowchart of an exemplary method to optimize the transducer of FIG. 1 for use in a therapy application, such as a HIFU Therapy, at a desired frequency;

FIG. 4 is a representative view of an exemplary system for characterizing the transducer of FIG. 1;

FIG. 5 is a flowchart of an exemplary method to determine a threshold power for the transducer of FIG. 1;

FIG. 6 is a flowchart of another exemplary method to optimize the transducer of FIG. 1 for use in a therapy application, such as a HIFU Therapy, at a desired frequency;

FIG. 7 is a flowchart of yet another exemplary method to optimize the transducer of FIG. 1 for use in a therapy application, such as a HIFU Therapy, at a desired frequency;

FIG. 8 is a flowchart of a further exemplary method to optimize the transducer of FIG. 1 for use in a therapy application, such as a HIFU Therapy, at a desired frequency;

FIG. 9 is a graphical representation of a plot of the total acoustic power of the transducer of FIG. 1 at the desired frequency of about 4.00 MHz as a function of lapping iteration;

FIG. 10 is a graphical representation of a plot of the excitation frequency corresponding to a maximum of the total acoustic power of the transducer of FIG. 1 as a function of lapping iteration; and

FIG. 11 is a three-dimensional graphical representation of the total acoustic power of the transducer of FIG. 1 as a function of lapping interval and excitation frequency.

### Detailed Description of the Drawings

In one embodiment, disclosed herein are exemplary apparatuses and methods for optimizing an acoustic transducer for therapy applications. In another embodiment, disclosed herein are exemplary apparatuses and methods for optimizing an acoustic transducer for therapy applications and for imaging applications simultaneously.

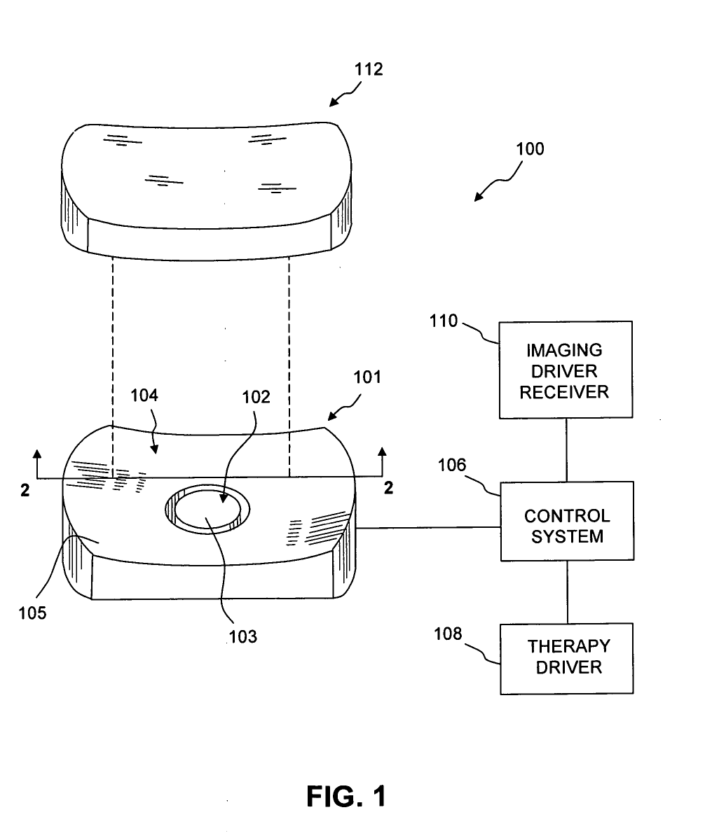

Referring to FIG. 1, a transducer 100 is shown. Transducer 100 includes a transducer member (“crystal”) 101 capable of emitting an acoustic signal. Transducer member 105 includes a first transducer element 102 and a second transducer element 104. In the illustrated embodiment, transducer element 102 is surrounded by transducer element 104. In one embodiment, a face 105 of transducer element 104 is spherical in shape and a face 103 of transducer element 102 is one of spherical in shape and aligned with transducer element 104 or generally flat.

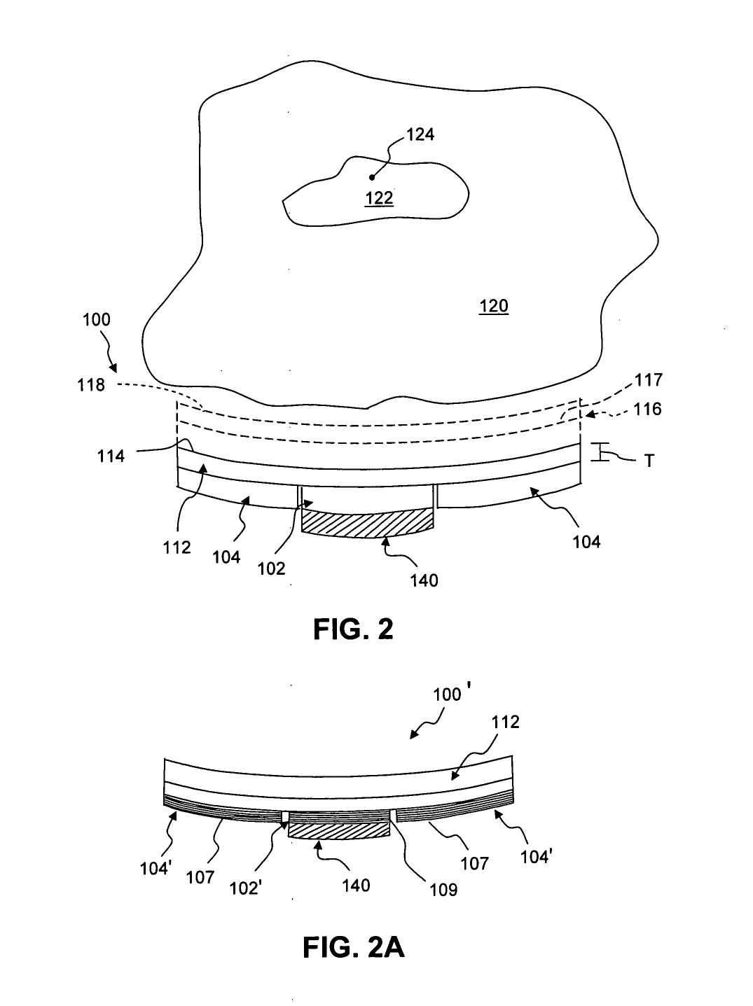

Transducer elements 102 and 104 are each individually drivable by a control system 106. In one embodiment, transducer element 104 is used in therapy applications and is driven by a therapy driver 108 to provide therapy, such as HIFU therapy, to portions of a surrounding environment 120 (see FIG. 2). In one embodiment, the surrounding environment 120 is tissue, such as the prostate 122. Transducer element 102 may also be used in therapy applications and is driven by therapy driver 108. Transducer element 102 may also be used in imaging applications and is driven by imaging driver and receiver 110.

In one embodiment, therapy driver 108 is configured to provide HIFU therapy. Exemplary HIFU therapy includes the generation of a continuous wave at a desired frequency for a desired time duration. In one example, the continuous wave is sustained for a period of time sufficient to ablate a target tissue at the desired location, such as a treatment site 124 within prostate 122. The location of treatment site 124 generally corresponding to the focus of transducer 100 which generally corresponds to the center of curvature of transducer 100. In one embodiment, control system 106 is configured to generate with therapy driver 108 a sinusoidal continuous wave having a frequency in the range of about 500 kHz to about 6 MHz, a duration in the range of about 1 second to about 10 seconds, with a total acoustic power at the focus in the range of about 5 Watts to about 100 Watts. In one example, the continuous wave is sinusoidal with a frequency of about 4.0 MHz and a duration of about 3 seconds. In another example, the continuous wave is sinusoidal with a frequency of about 4.0 MHz and a duration of about 3 seconds with a total acoustic power of about 37 Watts at the focus. This time period can be increased or decreased depending on the desired lesion size or the desired thermal dose. Further, the desired frequency may be adjusted.

Imaging driver and receiver 110 is configured to emit with transducer element 102 an imaging signal and to receiver echo acoustic energy that is reflected from features in the surrounding environment 120, such as prostate 122. The received signals may be used to generate one or more two-dimensional ultrasound images, three-dimensional ultrasound images, and models of components within the surrounding environment 120. Both therapy driver 108 and imaging driver and receiver 110 are controlled by control system 106. In addition, control system 106 may be further configured to utilize transducer element 102 for Doppler imaging of moving components within surrounding environment 120, such as blood flow. Exemplary imaging techniques including Doppler imaging are disclosed in PCT Patent Application Serial No. US2005/015648, filed May 5, 2005, designating the US, titled “Method and Apparatus for the Selective Treatment of Tissue”, Attorney Docket No. FOC-P001-01, the disclosures each of which is expressly incorporated herein by reference

Although transducer 100 is shown having a generally spherical shape and having two independently driven transducer elements 102 and 104, any transducer may be optimized with the apparatuses and methods disclosed herein. Exemplary transducers include single element transducers, multiple element transducers, and transducer arrays. Further, exemplary transducers may have a flat, a spherical, a cylindrical, an elliptical, or other suitable shape. In addition, the exemplary transducers may have a natural focus, and/or have one or more foci which are generated by electronically delaying the phase of various elements of the transducer.

Transducer 100 further includes a matching layer 112. Matching layer 112 is applied to faces 103 and 105 of transducer elements 102 and 104 as shown in FIG. 2. In one embodiment, matching layer 112 is an epoxy mixture applied to the face of transducer elements 102 and 104. In one example, matching layer 112 is an electrically resistant epoxy, such as Duralco™ 4525 available from Cotronics Corporation located at 3379 Shore Parkway, Brooklyn, N.Y. 11235. In one embodiment, transducer elements 102 and 104 are coated with a primer, such as AP 134 available from Bergdahl Associates located at 2990 Sutro St. Reno, Nev. USA 89512. In another embodiment, matching layer 112 is a polymer.

As discussed herein, matching layer 112 is altered such that transducer 100 is optimized for a transducer for use in a therapy application at a desired frequency. Referring to FIG. 2, one of the parameters of matching layer that may be altered to optimize transducer 100 is a thickness T (see FIG. 2) of matching layer 112. As explained herein, different thicknesses of matching layer 112 may result in different levels of power being delivered to the focus of transducer 100 for a given excitation frequency. However, a given thickness T of matching layer 112 may not be universally optimal for every transducer 100 because each transducer 100 is unique due to thickness variations in the crystals used for transducer elements 102 and 104 and other parameters, such as transducer crystal material variations, acoustical impedance, and the center/operating frequency. Also, variations might exist in the matching layer applied to two different transducers 100, such as thickness, density, or the speed of sound in the matching layer material. As such, a standard thickness of matching layer 112 applied to transducer member 101 does not guarantee that the transducer will be optimized for use in a therapy application at a desired frequency.

The methods disclosed herein are developed to take into account the individual characteristics of a given transducer 100. The following exemplary discussion is explained with transducer 100 which includes ceramic transducer elements 102 and 104. The ceramic transducer elements being available from Fuji Ceramic with overseas sales office located at Musashiya Bldg. 4F, 29-7, Kami-ochiai 1 -chome, Shinjuku-ku, Tokyo 161-0034. Transducer 100 is to be configured for use in therapy applications at a desired operating frequency of about 4 MHz. However, it should be appreciated that the following discussion is equally applicable to any transducer type, including single element transducers and multiple element transducers (made of individual, separate elements or a single crystal on which individual elements are defined via electrode patterns) and for any desired operating frequency. Referring to FIG. 2A a representative transducer 100′ is shown. Transducer 100′ includes a central element 102′ and an outer element 104′ whose separation is defined by the respective electrodes 109, 107 for each element on a convex back side of transducer 100′

Referring to FIGS. 2-4, an exemplary method of optimizing transducer 100 is shown. A extra thick layer of matching layer 112 is applied to the faces 103, 105 of transducer elements 102 and 104, as represented by block 202. Referring to FIG. 2, the extra thick layer of matching layer 112 is indicated by the dashed lines as extra thick matching layer 116. In one example about 0.2 mm to about 1 mm of matching layer is applied to the faces 103, 105 of transducer elements 102 and 104.

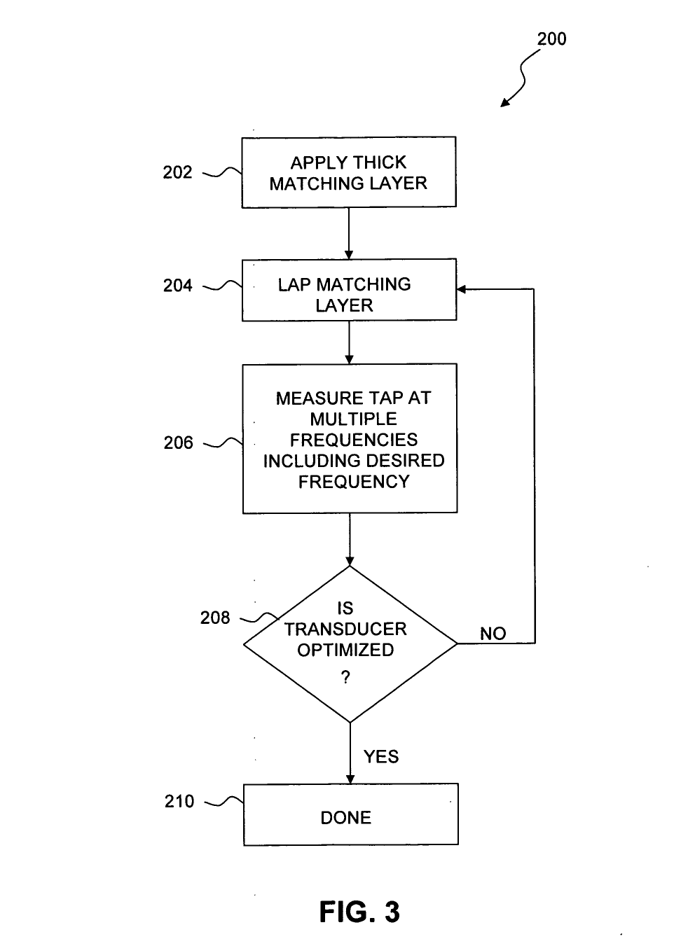

Returning to FIG. 3, a face 118 (see FIG. 2) of matching layer 116 is removed or lapped with a lapping machine, as represented by block 204. As is known in the art, lapping machines use lapping tools and/or lapping compound to grind away material or polish surfaces. Typically a lapping tool having the desired profile is used, in the case of transducer 100 a spherical tool having the same radius of curvature of transducer 100. Then the lapping tool and the transducer are brought into contact with each other and at least one of the lapping tool and the transducer are moved relative to the other. Exemplary lapping tools are available from Precision Tool Technologies, Inc. located at P.O. Box 56,309 NW13^{th} Avenue, Little Falls, Minn. 56345. It should be noted that matching layer 116 is not immediately removed down to face 114 of matching layer 112. On the contrary, the location of face 114 is generally not known and an iterative lapping process is carried out to determine the location of face 114 for a given instance of transducer 100. For illustrative purposes, face 118 is lapped down to a first intermediate face 117. It should be noted that each time the face of transducer is lapped, the lapping should be performed to provide a generally uniform thickness of matching layer 112.

In one embodiment, the uniformity of the thickness of matching layer 112 may be discemable from a plot of total acoustic power as a function of frequency. One indication of uniform thickness may be a generally symmetrical plot. Similarly, one indication of non-uniform thickness may be a generally non-symmetrical plot.

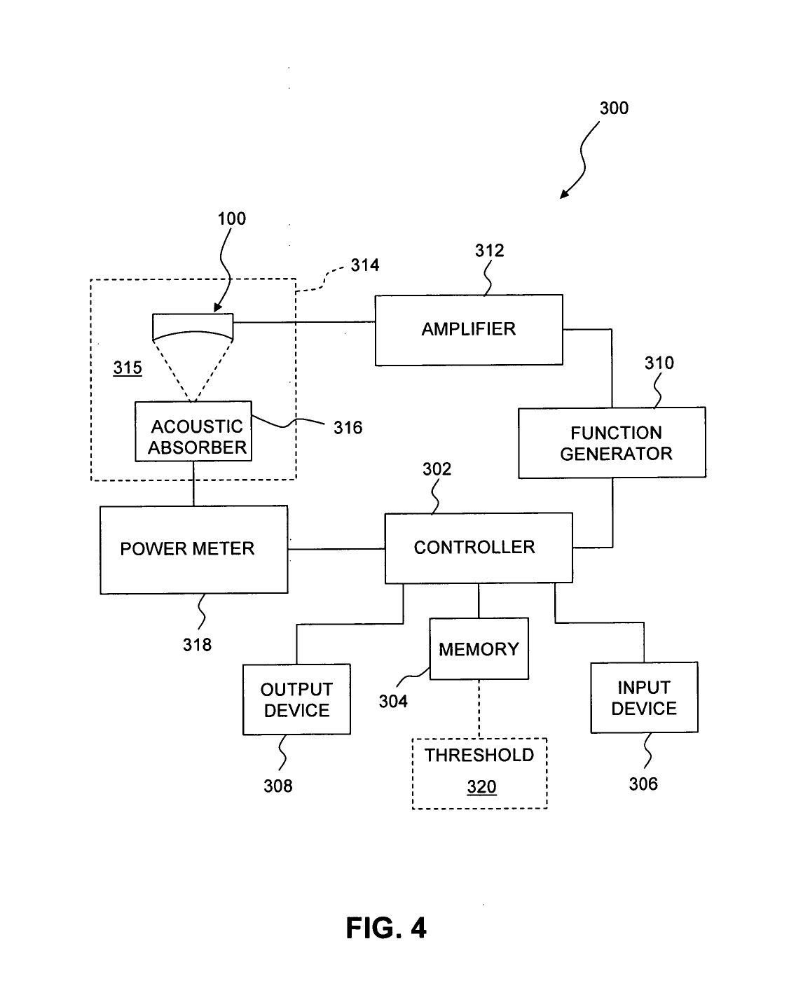

Next the acoustic performance of transducer 100 is tested for the given thickness of the matching layer, at intermediate level 117, as represented by block 206. Illustratively, the total acoustic power (“TAP”) generated by the transducer is monitored for various excitation frequencies. In one embodiment, the transducer is excited at various frequencies at an overall low transducer excitation level so as to not destroy or overly stress the non-optimal transducer member and matching layer assembly. In one example, about 10 percent to about 20 percent of the eventual maximum transducer excitation level is used for evaluating the transducer member and matching layer assembly during the matching layer optimization iterations. One apparatus for testing the performance of transducer 100 is shown in FIG. 4 and discussed below.

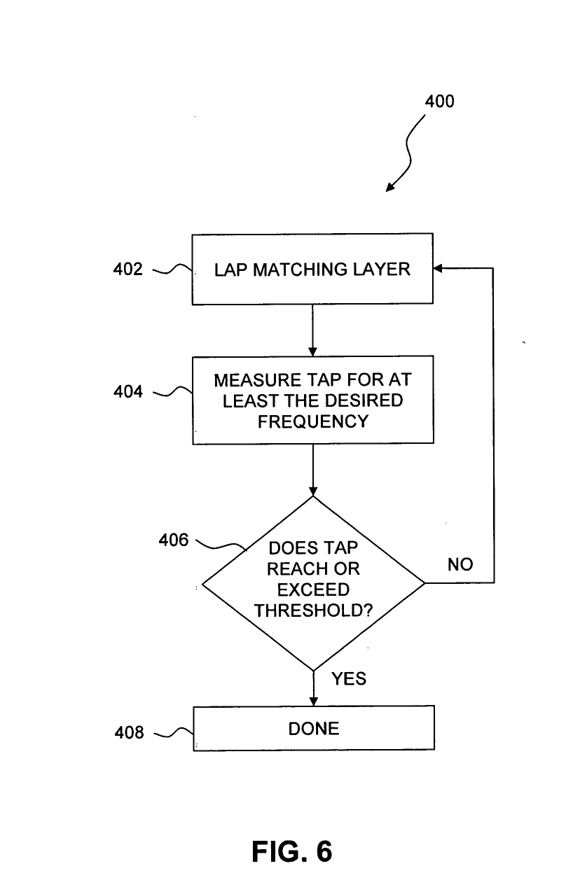

Based on the measured TAP values a decision is made on whether transducer 100 is optimized, as represented by block 208. If transducer 100 is optimized than the process is complete. If the transducer 100 is not optimized than additional material is removed from face 117 with the lapping machine and the performance of the further lapped transducer is tested. This process is repeated until transducer 100 is considered optimized. Exemplary methods for determining whether transducer 100 is optimized are shown in FIGS. 6-8.

Referring to FIG. 4, an exemplary test apparatus 300 is shown. Test apparatus 300 includes a controller 302 having an associated memory 304, an input device 306, and an output device 308. Memory 304 may be one or memory devices located locally or across a network. Exemplary input devices include one or more of a keyboard, a mouse, a trackball, a touch screen, a keypad, or other suitable input devices. Exemplary output devices include a display, a printer, or other suitable output devices.

Controller 302 includes software and/or hardware which is capable of performing the steps represented by blocks 206 and 208 in FIG. 3 and the exemplary methods discussed in connection with FIGS. 6-8. In one embodiment, controller provides a first indication, such as a message to the user that transducer 100 requires additional lapping. One exemplary message is “transducer total acoustic power output at desired frequency is sub-optimal.” In one embodiment, the user specifies through a user input device 306 the desired frequency and the message is provided to the user through an output device 308. In another embodiment, controller 302 provides a second indication, such as another message that transducer 100 is optimized. One exemplary message is “Maximum TAP at desired frequency is achieved.”

Controller 302 is operably coupled to and provides input to a function generator 310. Exemplary function generators include Model No. HP33120A available from Hewlett Packard located at 3000 Hanover Street Palo Alto, Calif. 94304- 1185. Function generator 310 is connected to or includes an amplifier 312 which is in turn operably coupled to transducer 100. Amplifier 312 amplifies the signal generated by function generator 310 to drive transducer 100. In one embodiment, transducer 100 is driven at a lower power than the power used for therapy applications. In one example, transducer 100 provides a total acoustic power of about 50 Watts to about 60 Watts and transducer 100 is tested with apparatus 300 with a total acoustic power of about 5 Watts to about 6 Watts in order to not overly stress and/or destroy the sub- optimal matching layer/transducer member assembly.

Transducer 100 is placed in a test vessel 314 containing a substance 315 which approximates the acoustic properties of the surrounding environment 120 which transducer 100 will be immersed in during a therapy application. Also included in the test vessel 314 is a sensor, illustratively an acoustic absorber 316, placed generally in the pre-focal zone of transducer 100. Absorber 316 absorbs the total acoustic power provided by transducer 100 and transfers the force associated with absorbing the acoustic energy to a coupled radiation force balance or scale of an acoustic power meter 318, calibrated in watts to measure the total acoustic power radiated by the transducer. An exemplary absorber and acoustic power meter is Model No. UPM-DT-10 available from Ohmic instruments, Company located in Easton, Md. 21601.

Controller 302 provides an input to function generator 310 to drive transducer 100 at a plurality of frequencies, each at spaced apart intervals of time, so that the total acoustic power of transducer 100 may be observed for each driving frequency independently. These observed total acoustic powers are stored by controller 302 in memory 304 for further processing to determine if transducer 100 is optimized for a desired frequency at the current thickness of matching layer 112.

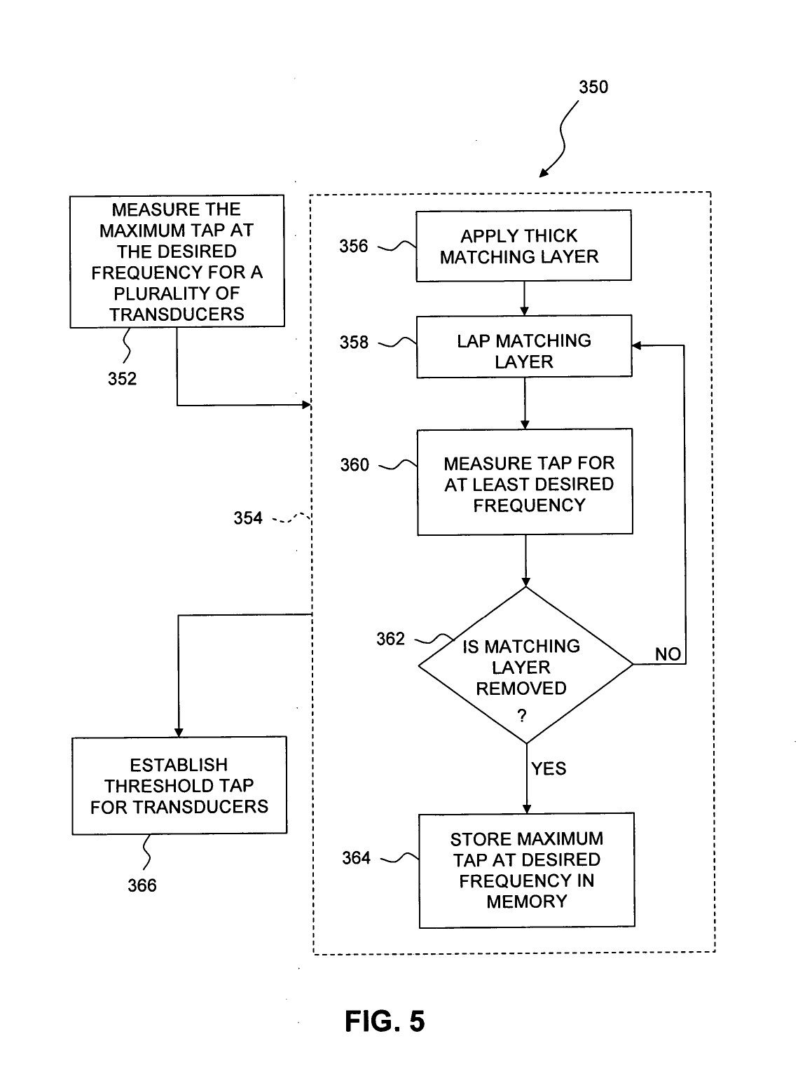

Referring to FIG. 5, in one exemplary method 350 a threshold total acoustic power 320 (see FIG. 4) is stored in memory 304. The performance characteristics of a plurality of transducers are monitored to determine the expected maximum TAP that a given transducer 100, generally similar to the plurality of transducers, should exhibit. As such, the maximum TAP at the desired frequency is measured for a plurality of transducers, as represented by block 352. An exemplary method of obtaining the maximum TAP for each transducer of the plurality of transducers is represented by block 354.

Referring to block 354, for each transducer of the plurality of transducers, an overly thick matching layer 112 is applied, as represented by block 356. The thickness of matching layer 112 is reduced and the shape of matching layer 112 is made generally concentric with transducer element 102, 104 by lapping matching layer 112 with a suitable shaping tool, as represented by block 358. Next the TAP of transducer 100 is measured with apparatus 300 for at least the desired frequency, as represented by block 360. In one embodiment, the TAP of transducer 100 is measured with apparatus 300 for a plurality of frequencies to provide trending information, such as if the maximum TAP is migrating towards or away from the desired frequency for successive iterations of lapping. This process is repeated until the entire matching layer is removed, as represented by block 362. Once the matching layer 112 has been completely lapped, the maximum TAP at the desired frequency is recorded in memory 304, as represented by block 364.

As stated above, this process is repeated for a plurality of transducers to empirically determine what measured maximum TAP corresponds to a typical maximum TAP output power (i.e. the expected maximum TAP, or the TAP at which the transducer is deemed acceptable). As such, a threshold TAP for future instances of similar transducers is determined, as represented by block 366.

In one embodiment, the threshold is determined by the desired yield of the manufacturing operation. In one example, the TAP of the transducer 100 at the desired frequency must be equal to or greater than the TAP achieved by the best 50% of the transducers characterized with the process of FIG. 5.

Referring to FIG. 6, an exemplary method 400 of employing the threshold established in the above mentioned method is illustrated. For a given transducer 100, matching layer 112 is lapped, as represented by block 402. The TAP for at least the desired frequency is measured, as represented by block 404, such as with apparatus 300. If the measured TAP at the desired frequency meets or exceeds the threshold, then transducer 100 is considered optimized, as represented by blocks 406 and 408. Otherwise, transducer 100 is lapped at least one more time, as indicated by the connection between blocks 406 and 402.

In another embodiment, as the TAPs of the plurality of transducers are detected in FIG. 5, an input electrical impedance of transducer 100 is monitored with an impedance analyzer. An exemplary impedance analyzer is Model No. 4194A available from Hewlett Packard located at 3000 Hanover Street Palo Alto, Calif. 94304-1185. Based on a determination of a threshold input impedance value future instances of transducer 100 may be analyzed without the need of monitoring the TAP of the transducer. Rather, the input impedance of the given instance of the transducer may be monitored. In one embodiment, the threshold impedance value is determined by the desired yield of the manufacturing operation. In one example, the TAP of the transducer 100 at the desired frequency must be equal to or greater than the TAP achieved by the best 50% of the transducers characterized.

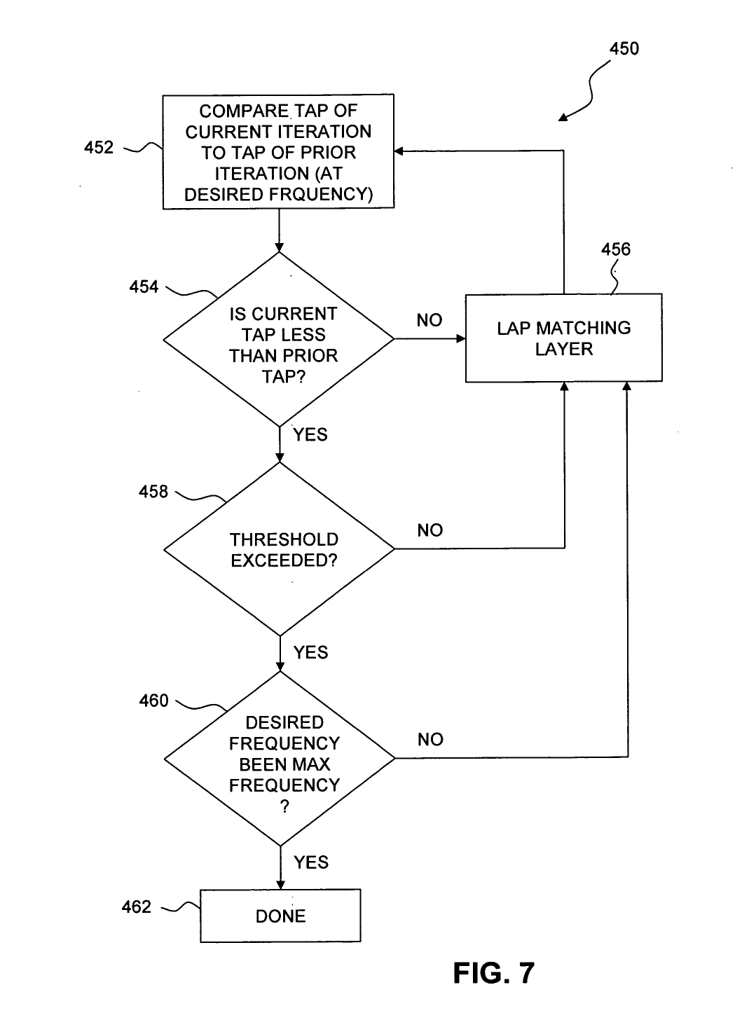

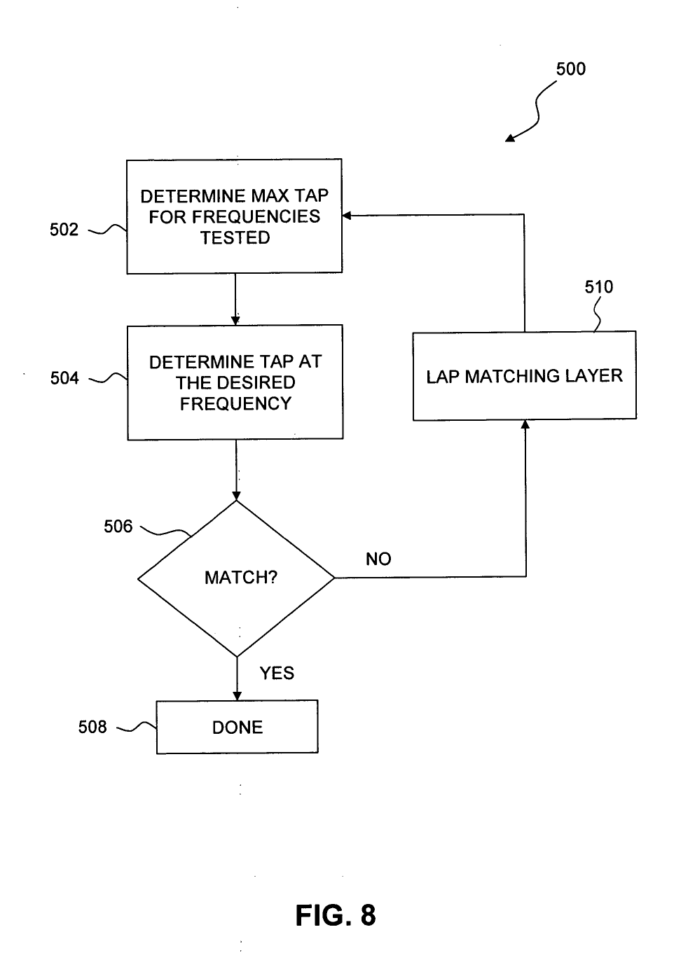

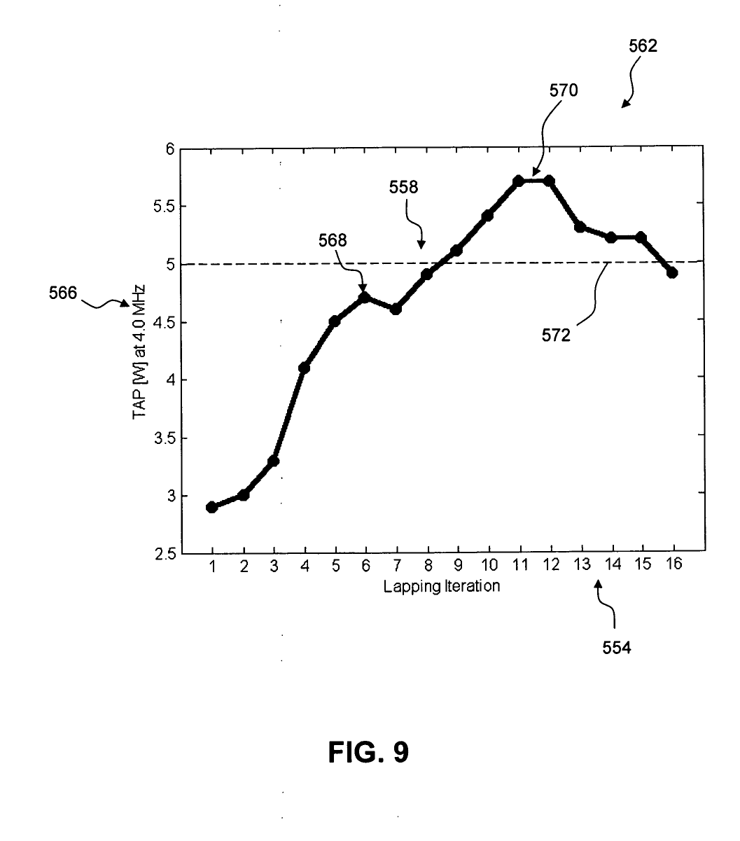

Two further exemplary methods, 450 and 500, of determining if transducer 100 is optimized are explained below in connection with FIGS. 7 and 8, respectively. Both of these methods are explained with the aid of an exemplary optimization of an exemplary transducer, illustratively shown in FIGS. 9-11. These methods have the advantage of not requiring the previous characterization of many transducers to establish a pass/fail threshold, but rely on trends that appear during the lapping iterations to determine if the transducer/matching layer has been optimized. As such, these methods are well suited for custom or non-mass produced transducers as well as mass produced transducers. Referring to FIGS. 9-11, sixteen lapping iterations are shown for illustrative purposes. It should be noted that the exemplary transducer was optimized at lapping iteration 12. However, additional lapping was carried out to aid in the illustration of the methods in FIGS. 7 and 8.

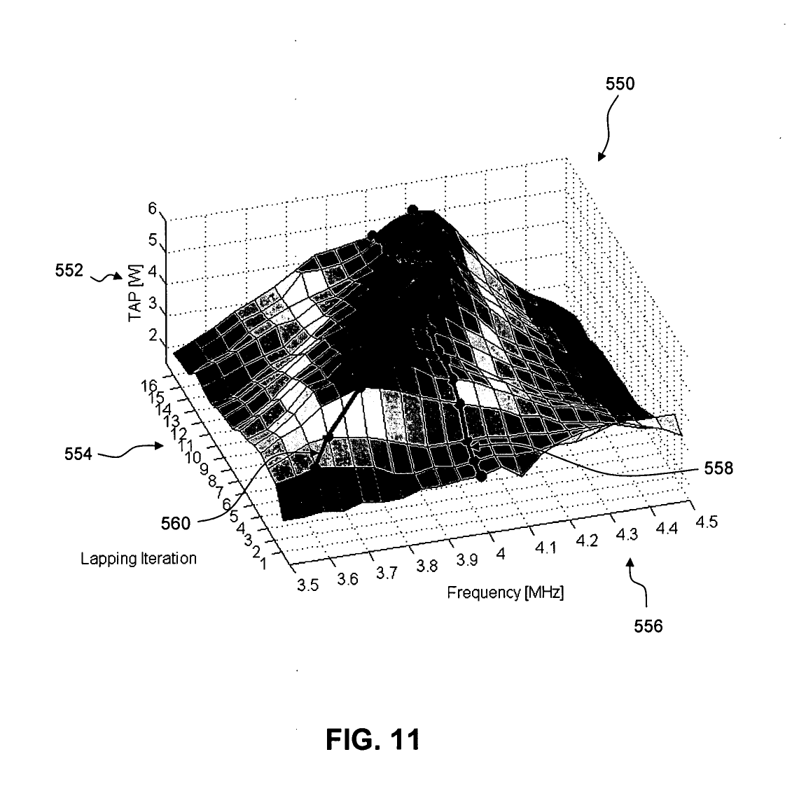

Referring to FIG. 11, a plot 550 is shown plotting measured TAP values 552 as a function of lapping iteration 554 and as a function of excitation frequency 556. As may be seen in FIG. 11, for each lapping iteration a plurality of excitation frequencies are tested, illustratively excitation frequencies spanning from about 3.55 MHz to about 4.50 MHz at 50 kHz intervals.

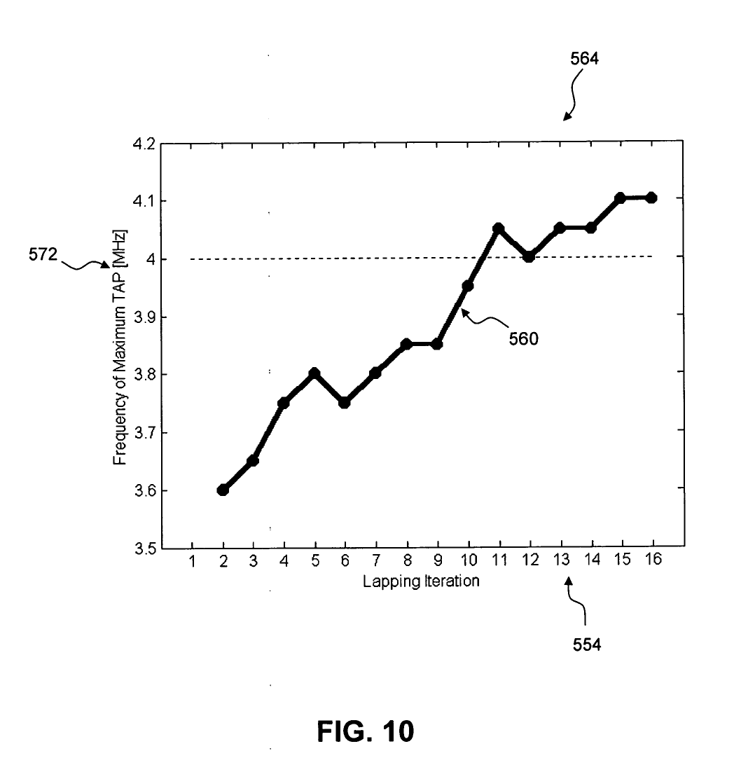

Curves 558 and 560 are indicated in FIG. 11 and are provided as two dimensional plots 562 and 564 in FIGS. 9 and 10, respectively. The data point for the first lapping iteration of curve 560 is not shown due to an artifact in data. Referring to FIG. 9, plot 562 illustrates measured TAP values at the desired frequency of about 4.00 MHz 566 as a function of lapping interval 554. As may be seen in plot 562, the measured TAP values have a first (“local”) maximum 568 corresponding to lapping iteration 6 and a second (“global”) maximum 570 corresponding to lapping iterations 11 and 12. Referring to FIG. 10, plot 564 illustrates the frequency corresponding to the maximum measured TAP 572 as a function of lapping interval 554. As may be seen in plot 564, the desired frequency of about 4.00 MHz corresponds to the maximum measured TAP value for lapping iteration 12.

Referring to FIG. 7, in exemplary method 450, for a given lapping iteration the measured TAP for the desired frequency of the current iteration is compared to the measured TAP for the desired frequency of the prior iteration, as represented by block 452. If the current measured TAP is greater than the prior measured TAP, the matching layer of the transducer is lapped again, as represented by blocks 454 and 456. If the current TAP is less than the previous TAP, this may be an indication that the transducer is optimized. In one embodiment, transducer 100 is considered optimized at this point (if no threshold information is available, such as in the case of a custom transducer).

However, in the illustrated embodiment, further processing is performed to further verify if transducer 100 is optimized. Two additional exemplary criteria or tests are discussed in connection with blocks 458 and 460. Referring to block 458, the current measured TAP is compared to a threshold TAP for the transducer being tested. In one embodiment, this threshold TAP is calculated as discussed above. If the threshold TAP has not been met or exceeded, the matching layer of the transducer 100 is further lapped, as represented by block 456. Referring to block 460, a check is made whether the desired frequency has been the frequency corresponding to the maximum TAP in either the current iteration or prior iterations. If not, then the matching layer of transducer 100 is further lapped. If so, transducer 100 is determined to be optimized.

Referring to FIGS. 9 and 10 and stepping through exemplary method 450, the matching layer of the transducer will be directed for further lapping at block 454 until lapping iteration 7 is analyzed. At lapping iteration 7, the current TAP is less than the prior TAP, lapping iteration 6, and the process advances to block 458, wherein the current TAP is compared to a threshold TAP, illustratively shown as line 572 in FIG. 9 and illustratively corresponding to about 5 W (about 50 W for therapy assuming testing being carried out at 10% of therapy power). Since the current TAP of about 4.5 W is less than the threshold TAP of about 5.0 W, the matching layer of the transducer is further lapped. In one embodiment, block 458 is not included in the exemplary embodiment and block 454 passes directly to block 460. In another embodiment, block 458 is the final criteria and block 458 passes directly to block 462.

Lapping of the matching layer of transducer is repeated by block 454 until lapping iteration 13 is analyzed. At lapping iteration 13, the current TAP is less than the prior TAP so block 454 passes onto block 458. At block 458, the current TAP exceeds the threshold TAP, so block 458 passes onto block 460. At block 460, the desired frequency (about 4.00 MHz) had been the frequency corresponds to the maximum frequency for iteration 12 (see FIG. 10). As such, transducer 100 is determined to be optimized.

Referring to FIG. 8, transducer 100 is considered optimized based on a analysis of lines 558 and 560, such as looking for an intersection of lines 558 and 560 (see FIG. 11). For a given iteration, the maximum measured TAP is determined for the frequencies tested, as represented by block 502. Over time this is represented by line 560 in FIG. 11. Further, the measured TAP at the desired frequency is determined, as represented by block 504. Over time expressed as lapping iterations this is represented by line 558 in FIG. 11. These two TAP values are compared and if equal transducer 100 is considered optimized, as represented by blocks 506 and 508 in FIG. 8. If the two values are unequal, then transducer 100 is further lapped as represented by block 510.

Referring to FIG. 11, exemplary method 500 would be carried out until iteration 12 wherein the measured TAP at the desired frequency is equal to the maximum measured TAP across the monitored frequencies. It should be noted that instead of comparing TAP values corresponding to frequencies, in one embodiment the frequencies corresponding to the TAP values may be compared.

Method 500 is able to distinguish local maxima from global maxima without the need of first calculating as threshold TAP value, as used in exemplary method 450. As may be seen in FIG. 11, method 500 does not stop at local maxima in the TAP at the desired frequency corresponding to iteration 6 because the frequency corresponding to the maximum TAP is about 3.80 MHz not about 4.00 MHz.

It should be noted that the methods for analyzing the TAP values for transducer 100 at various excitation frequencies disclosed herein may be performed by controller 302, by an operator observing the TAP data, such as one or more of the graphs shown in FIGS. 9-11, or a combination thereof.

In one embodiment transducer 100 is further optimized for use in imaging applications. As stated above transducer element 102 may be used in both therapy applications and imaging applications. However, the alteration of matching layer 1 12 discussed herein has been designed to optimize transducer 100 for therapy applications, not imaging applications.

To state it another way, transducer 100 has been optimized (matching layer 1 12) for a narrow bandwidth or frequency band around the desired frequency and is air-backed, whereas a traditional imaging transducer is optimized to have a wide bandwidth over a broad range of frequencies and is matched on the front surface and damped in the back to eliminate ringing. As is, transducer 100 is not optimized for imaging applications and may exhibit lower amplitude signals and ringing phenomenon if the central element is used imaging applications. Referring to FIG. 2, the imaging ability of transducer element 102 may be improved to compensate for the overall/global therapy optimization of matching layer 1 12 by placing a thicker/heavier backing 140 on transducer element 102 than traditionally employed if the transducer matching layer were optimized via standard imaging optimization only. In one embodiment, backing 140 is about 1 mm to about 2 mm thick and is made of 4538 epoxy. The density of the epoxy may be further increased, for example, by adding tungsten powder of various mesh sizes to achieve a higher density. The heavier the backing is the more damping provided by the backing 140. The heaviness of backing 140 may be increased by either increasing the thickness of backing 140 and/or increasing the density of backing 140.

Although the invention has been described in detail with reference to certain preferred embodiments, variations and modifications exist within the spirit and scope of the invention as described and defined in the following claims.

1. A method of optimizing an ultrasound transducer to provide therapy with HIFU at a desired frequency, the method comprising the steps of:
driving an ultrasound transducer at a plurality of acoustic frequencies;
detecting an acoustic power of the ultrasound transducer for each of the plurality of acoustic frequencies; and
altering a thickness of a matching layer of the ultrasound transducer based on the detected acoustic power of the ultrasound transducer.

2. The method of claim 1, wherein the step of altering the thickness of the matching layer is repeated until a maximum detected acoustic power is at the desired frequency.

3. The method of claim 1, wherein the step of altering the thickness of the matching layer is repeated until a stored threshold power is exceeded by the detected acoustic power at the desired frequency.

4. The method of claim 1, wherein the step of detecting the acoustic power of the ultrasound transducer includes the steps of:
placing the ultrasound transducer in a test tank filled with a test substance;
placing an acoustic absorber in a position to receive the acoustic energy of the ultrasound transducer; and
sensing the acoustic power of the ultrasound transducer for each acoustic frequency with the acoustic absorber to determine a total acoustic power output of the ultrasound transducer for each acoustic frequency.

5. The method of claim 1, wherein the ultrasound transducer includes a central transducer element and an outer transducer element and wherein the method further comprises the step of applying a backing to at least the central transducer element.

6. The method of claim 5, further comprising the step of adjusting one of a thickness of the backing and a density of the backing to optimize the central element for an imaging mode of operation.

7. A method of optimizing an ultrasound transducer to provide therapy with HIFU at a desired frequency, the method comprising the steps of:
receiving an indication of an acoustic power of the ultrasound transducer across a range of acoustic frequencies, including the desired frequency; and
altering a thickness of a matching layer of the ultrasound transducer until a maximum of the acoustic power of the ultrasound transducer across the range of acoustic frequencies corresponds to the desired frequency.

8. The method of claim 7, further comprising the step of applying the matching layer to a face of a transducer member of the ultrasound transducer, the matching layer having an initial thickness greater than a final optimized thickness.

9. The method of claim 8, wherein the step of altering the thickness of the matching layer comprises the steps of:
(a) lapping a face of the matching layer to reduce the thickness of the matching layer;
(b) receiving an updated indication of the acoustic power of the ultrasound transducer across the range of acoustic frequencies; and
(c) repeating steps (a) and (b) until the maximum of the acoustic power of the ultrasound transducer corresponds to the desired frequency.

10. The method of claim 7, wherein the indication of the acoustic power of the ultrasound transducer is received by the steps of:
placing the ultrasound transducer in a test tank filled with a test substance;
placing an acoustic absorber in a position to receive the acoustic energy of the ultrasound transducer;
exciting the ultrasound transducer to generate a plurality of acoustic frequencies, each at a respective period of time; and
sensing the acoustic power of the ultrasound transducer for each acoustic frequency with the acoustic absorber to determine a total acoustic power output of the ultrasound transducer for each acoustic frequency.

11. The method of claim 7, wherein the indication of the acoustic power of the ultrasound transducer is determined by the step of monitoring an impedance associated with the ultrasound transducer.

12. The method of claim 7, wherein the ultrasound transducer includes a central transducer element and an outer transducer element and wherein the method further comprises the step of applying a backing to at least the central transducer element.

13. The method of claim 12, further comprising the step of adjusting one of a thickness of the backing and a density of the backing to optimize the central element for an imaging mode of operation.

14. A method of optimizing an ultrasound transducer to provide therapy with HIFU at a desired frequency, the method comprising the steps of:
(a) detecting a plurality of acoustic powers of the ultrasound transducer, each acoustic power corresponding to a given frequency in a range of acoustic frequencies, including the desired frequency;
(b) altering a thickness of a matching layer of the ultrasound transducer; and
(c) repeating steps (a) and (b) until a detected power corresponding to the desired frequency for a current iteration is less than a detected power corresponding to the desired frequency for a prior iteration.

15. The method of claim 14, wherein steps (a) and (b) are repeated until the detected power corresponding to the desired frequency for the current iteration is less than the detected power corresponding to the desired frequency for the prior iteration and the detected power corresponding to the desired frequency for the current iteration exceeds a threshold power level.

16. The method of claim 14, wherein steps (a) and (b) are repeated until the detected power corresponding to the desired frequency for the current iteration is less than the detected power corresponding to the desired frequency for the prior iteration, the detected power corresponding to the desired frequency for the current iteration exceeds the threshold power level, and the detected power corresponding to the desired frequency for the current iteration is equal to a maximum detected power for the current iteration.

17. The method of claim 14, further comprising the step of applying the matching layer to a face of a transducer member of the ultrasound transducer, the matching layer having an initial thickness greater than a final thickness.

18. The method of claim 14, wherein the step of detecting a plurality of acoustic powers of the ultrasound transducer includes the steps of:
placing the ultrasound transducer in a test tank filled with a test substance;
placing an acoustic absorber in a position to receive the acoustic energy the ultrasound transducer;
exciting the ultrasound transducer to generate a plurality of acoustic frequencies, each at a respective period of time; and
sensing the acoustic power of the ultrasound transducer for each given acoustic frequency with the acoustic absorber to determine a total acoustic power output of the ultrasound transducer for each given acoustic frequency.

19. The method of claim 14, wherein the transducer includes a central transducer element and an outer transducer element and wherein the method further comprises the step of applying a backing to at least the central transducer element.

20. The method of claim 19, further comprising the step of adjusting one of a thickness of the backing and a density of the backing to optimize the central element for an imaging mode of operation.

21. A computer readable medium providing instructions for directing a controller to:
drive an ultrasound transducer at a plurality of acoustic frequencies;
detect an acoustic power of the ultrasound transducer for each of the plurality of acoustic frequencies; and,
provide an indication that a thickness of a matching layer of the ultrasound transducer be altered based on the detected acoustic power of the ultrasound transducer.

22. The computer readable medium of claim 21, wherein the indication is provided until a maximum detected acoustic power is at a frequency which is the desired frequency.

23. The computer readable medium of claim 21, wherein the indication is provided until a stored threshold power is exceeded by the detected acoustic power at the desired frequency.

24. The computer readable medium of claim 21, further comprising providing instructions to provide a second indication that transducer is optimized for a therapy application.
